(19) United States
(12) Patent Application Publication
(10) Pub. No.: US 2015/0065868 A1
(43) Pub. Date: Mar. 5, 2015

(54) SYSTEM, METHOD, AND COMPUTER ACCESSIBLE MEDIUM FOR VOLUMETRIC TEXTURE ANALYSIS FOR COMPUTER AIDED DETECTION AND DIAGNOSIS OF POLYPS

(71) Applicant: The Research Foundation for the State of University of New York, Stony Brook, NY (US)

(72) Inventors: Jerome Zheng Liang, Stony Brook, NY (US); Hongbing Lu, Stony Brook, NY (US); Guopeng Zhang, Stony Brook, NY (US)

(73) Assignee: The Research Foundation for the State University New York, Stony Brook, NY (US)

(21) Appl. No.: 14/390,212

(22) PCT Filed: Mar. 15, 2013
(86) PCT No.: PCT/US13/32110

§ 371 Date: Oct. 2, 2014

Related U.S. Application Data
(60) Provisional application No. 61/619,208, filed on Apr. 2, 2012.

(51) Int. Cl. G06T 7/00; G06T 7/60; A61B 6/00; A61B 6/12; A61B 6/03; A61B 5/00

(52) U.S. Cl. CPC G06T 7/0012; A61B 6/032; A61B 5/4255; A61B 6/5229; A61B 6/12; G06T 7/604; G06T 2207/10081; G06T 2207/30032
USPC 600/425; 382/131

(57) ABSTRACT

A computer-based method for diagnosing a region of interest within an anatomical structure having the steps of receiving a 3D volumetric representation of the anatomical structure, and identifying at least one volume of interest and volume of normal of the anatomical structure. A first feature set can be generated based on a density, a gradient and a curvature of the volume of interest, and the first feature set can be compared to a second feature set to diagnose the region of interest to at least one a plurality of pathology types.

### CROSS-REFERENCE TO RELATED APPLICATIONS

The present application claims the benefit under 35 U.S.C. §119(e) of U.S. Provisional Patent Application Ser. No. 61/619,208 filed on Apr. 2, 2012, the content of which is hereby incorporated by reference in its entirety.

### STATEMENT OF GOVERNMENT RIGHTS

This invention was made with government support under grant number CA082402 awarded by the National Institute of Health. The government has certain rights in the invention.

### FIELD OF THE DISCLOSURE

The present disclosure relates generally to a computer aided diagnosis of diseases, and more specifically, relates to the detection and diagnosis of colonic polyps using in vivo imaging.

### BACKGROUND INFORMATION

Colorectal carcinoma is the third most common cancer in both men and women worldwide. According to the American Cancer Society, an estimated 101,340 cases of colon cancer and 39,870 cases of rectal cancer are expected to occur in 2011. Colorectal cancer incidence rates have been decreasing for the past two decades, from 66.3 cases per 100,000 persons in 1985 to 45.3 in 2007. The declining rate accelerated from 1998 to 2007 (e.g., 2.9% per year in men and 2.2% per year in women), which can be attributed to the increase in the use of colorectal cancer screening tests that allow the detection and removal of colorectal polyps before they can progress to cancer. In contrast to the overall decline, among younger adults less than 50 years old who are not at the average risk and for which the screening is not recommended, the colorectal cancer incidence rate has been increasing by 1.6% per year since 1998. This indicates that it may be beneficial to perform regular screening examinations by a healthcare professional, which can result in the detection and removal of precancerous growths at an early stage when the growths are most treatable. Fiber-optical colonoscopy (“FOC”) is currently the preferred method for colon polyp detection. However, the perceived discomforts associated with preparing the colon, and the potential proliferation risk, have impeded the usage of FOC. As a potential minimally-invasive screening technique, computed tomographic colonography (“CTC”), or CT-based virtual colonoscopy, has shown several advantages over a FOC. Computer-aided detection (“CADe”) of polyps has been proposed to improve the consistency and sensitivity of CTC interpretation, and to reduce interpretation burden.

A typical CADe pipeline for CTC starts from a three-dimensional (“3D”) model of the colon generated from the 2D CT data. From the 3D model, a segmented colon wall can be derived. Based on the segmented colon wall, initial polyp candidates (“IPCs”), each of which can be represented by a group of image voxels, namely a patch, can be localized on the colon wall. Unfortunately, due to the complexity of the colon structure (e.g., folds, cecal valve, etc.), and the presence of colonic materials such as fluid and fecal residuals which can mimic the structure of polyps, there can be a substantial number of false positives (“FP”) in the IPC pool. Therefore the reduction of the FP rate remains a challenge for the current CADe pipelines.

To achieve a high FP reduction rate, a large number of features have been developed to classify the IPCs. Combined with some empirical constraints, these features have worked well in differentiating some spherical IPCs from colon folds, which are a major source of FP findings in the colon. These features can be divided into two categories. The first category is geometry-related features that consider the global or local shape variations of the IPCs, such as shape index (“SI”), curvedness, sphericity ratio, convexity or concavity and surface normal overlap. These geometric features can generally be based on the assumption that there exists an iso-surface between a polyp and its neighboring tissues. Accurately detecting geometric features generally requires good quality image segmentation before the feature extraction procedure. The second category of features is texture-related features which typically consider the internal structure pattern of each IPC volume, such as gradient concentration, growth ratio, density projection, and statistical indices of the aforementioned features. For example, Yoshida and Nappi computed the values of some volumetric features (e.g., SI, curvedness, CT density value, gradient, gradient concentration (“GC”), and two GC-evolved features of dGC and mGC from all the image voxels in each IPC volume). (Nappi J. and Yoshida H., “Feature-guided analysis for reduction of false positives in CAD of polyps for computed tomographic colonography”, Medical Physics, 30(7): 1592-1601, 2002). The distribution of the values of each feature over the 3D space can form an internal pattern (e.g., volumetric texture) for each IPC. These volumetric textures can be depicted by some statistic indices of the distribution. Their results can indicate that these internal textural features have improved the performance of the CADe for CTC. The advantage of volumetric textural features over geometry-related features is that they can make full use of the image voxels inside an IPC volume to identify some internal patterns.

Based on the above, it may be beneficial to depict the texture features of an IPC volume and utilize the model for FP reduction in CADe. It may further be beneficial to perform computer aided diagnosis (“CADx”) for differentiating among pathology types of the various polyps detected by CADe.

### SUMMARY OF EXEMPLARY EMBODIMENTS

These and other deficiencies can be addressed with the exemplary systems, methods, and computer-accessible mediums for the detection and diagnosis of polyps set forth in the present disclosure.

These and other objects of the present disclosure can be achieved by provision of systems, methods and computer-accessible mediums for diagnosing a region of interest (“ROI”) within an anatomical structure which can include receiving a 3D volumetric representation of the anatomical structure, identifying at least one volume of interest (“VOI”) of the anatomical structure, generating a first feature set based on a density, a gradient and a curvature of the volume of interest, and comparing the first feature set to a second feature set to diagnose the region of interest to at least one of a plurality of pathology types.

In some exemplary embodiments, the generation of the first feature set can include determining a gradient of the volume of interest, determining a curvature of the volume of interest, and combining the gradient and the curvature with the original density to produce the first feature set. In some exemplary embodiments, at least one of the gradient or the curvature or the original density can be determined using a 3D Haralick model. In some exemplary embodiments, the gradient can be determined using a gray-level gradient co-occurrence matrix. In certain exemplary embodiments, the curvature can be determined using a gray-level curvature co-occurrence matrix. In certain exemplary embodiments, the original density can be determined using a gray-level co-occurrence matrix. In certain exemplary embodiments, the second feature set can be generated by manually analyzing a plurality of regions of interest. In certain exemplary embodiments, the anatomical structure can be a polyp. In some exemplary embodiments, the region of interest can be detected. In some exemplary embodiments, the region of interest is diagnosed only if the anatomical structure is detected to be a polyp. In certain exemplary embodiments, the detection can include comparing the volume of interest to the volume of normal (“VON”) in the 3D volumetric representation of the anatomical structure.

In certain exemplary embodiments, the 3D volumetric representation of the anatomical structure can be generated using an in-vivo imaging method. In certain exemplary embodiments, the first feature set can be compared to the second feature set using a support vector machine (“SVM”). In certain exemplary embodiments, 2D imaging information of the anatomical structure can be received and converted into the 3D volumetric representation of the anatomical structure. In certain exemplary embodiments, the 2D imaging information can be generated using computed tomography. In a preferred embodiment, the first feature set and the second feature set have at least 50 features.

In accordance with a further exemplary embodiment are systems, methods and computer-accessible mediums for diagnosing a region of interest within an anatomical structure which can include receiving first information related to a gradient and a curvature of a volume of interest and a volume of normal of the anatomical structure, and comparing the first information to the second information to diagnose the region of interest to be at least one of a plurality of pathology types.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a simplified flow diagram that illustrates an exemplary method for detecting and diagnosing a region of interest according to an exemplary embodiment of the present disclosure;

FIGS. 2(a)-(c) are exemplary images with manually-drawn outlines of the boundaries of the VOI and VON on the image slices;

FIG. 3 is an exemplary flow diagram illustrating an automatic segmentation procedure to refine the manually-drawn outlines of FIGS. 2(a)-(c) for the final boundaries of the VOI and VON according to an exemplary embodiment of the present disclosure;

FIG. 4 is an exemplary 3D model illustrating a transformation from 2D into 3D according to an exemplary embodiment of the present disclosure;

FIG. 5 illustrates an exemplary evaluation procedure according to exemplary embodiments of the present disclosure;

FIG. 6 is an exemplary image illustrating a global thresholding strategy according to exemplary embodiments of the present disclosure;

FIG. 7 is an exemplary graph illustrating a principal component analysis of Volume of Interest derived features according to an exemplary embodiment of the present disclosure;

FIG. 8 is an exemplary graph illustrating an exemplary accumulative variance curve according to an exemplary embodiment of the present disclosure;

FIG. 9 is an illustration of an exemplary block diagram of an exemplary system in accordance with certain exemplary embodiments of the present disclosure; and

FIG. 10 is an exemplary flow diagram illustrating a method for extracting a feature set.

Throughout the drawings, the same reference numerals and characters, unless otherwise stated, are used to denote like features, elements, components, or portions of the illustrated embodiments. Moreover, while the present disclosure will now be described in detail with reference to the Figures, it is done so in connection with the illustrative embodiments and is not limited by the particular embodiments illustrated in the Figures.

### DETAILED DESCRIPTION OF THE DISCLOSURE

The exemplary embodiments of the present disclosure may be further understood with reference to the following description and the related appended drawings. The exemplary embodiments of the present disclosure relate to exemplary systems, methods and computer-accessible mediums for the computer-aided detection and diagnosis of polyps in a virtual colonoscopy. Specifically, the exemplary system, method and computer-accessible medium can extract features of a region of interest, classify the features, and compare the features to a known feature set in order to detect and diagnose a polyp.

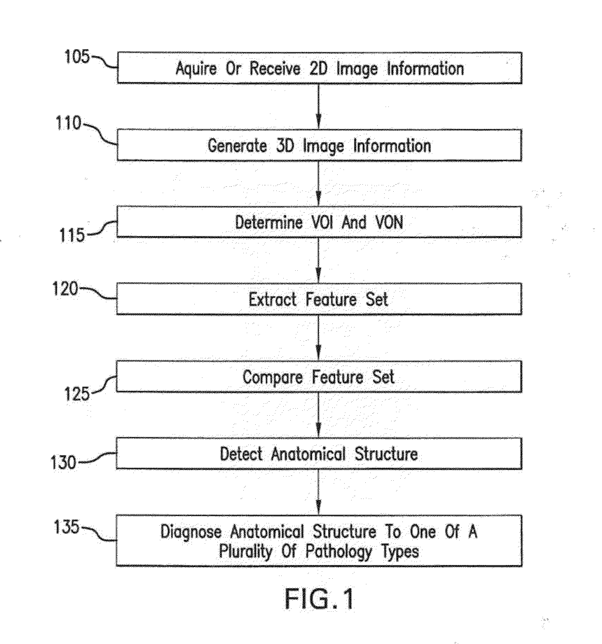

FIG. 1 is a flow chart according to an exemplary embodiment of the present system, method and computer-accessible medium for detecting and diagnosing polyps. At step 105, 2D imaging information, such as conventional CT data, can be acquired or received. For example, a plurality of images using an in-vivo imaging method (e.g., computed tomography) can be taken of the anatomical structure to be examined (e.g., the colon). The 2D information can be converted into 3D volumetric imaging information at step 110, using known techniques used in conventional virtual colonoscopy. It should be noted that the 2D imaging and 3D conversion can take place prior to the exemplary method, and the 3D volumetric model generated using conventional methods can be received and manipulated. For example, the 2D imaging information can be generated when a patient is being imaged, and the 3D conversion can take place after the 2D imaging information has been generated. Therefore, according to exemplary embodiments of the present disclosure, the 3D imaging information can be received and/or manipulated at a later time than the acquisition or generation of the 2D imaging information and/or the 3D imaging information.

After the 3D imaging information is generated and/or received, one or more volume(s) of interest within the anatomical structure being analyzed can be selected and corresponding volume(s) of normal can also be selected at step 115. A VOI can be selected based on IPCs in the 3D volumetric information, each of which can be represented by a group of image voxels. VONs can be selected based on each VOI, at a certain local distance away from the VOI. Based on the VOIs and the VONs, a feature set of the VOIs and VONs can be extracted at step 120, and compared to a further, known, feature set at step 125. The further feature set can be stored in a database, and can be generated based on a prior automatic or manual classification of real-world, non-virtual, biopsies.

### Exemplary Image Interpolation and Segmentation

The CT images can be acquired using the different image slice thickness from patient to patient. In order to facilitate the feature extraction, it can be beneficial to interpolate the CT images into the same image slice thickness for all patients. t For that purpose, each volumetric data of a CTC scan can undergo a known Monotonic Cubic Interpolation procedure to transform the image elements into isotropic cubic voxels, as described in Fritsch. (Fritsch F and Carlson R. “Monotone piecewise cubic interpolation”, SIAM Journal on Numerical Analysis (SIAM), 17(2): 238-246, 1980, the disclosure of which is hereby incorporated by reference in its entirety.) Because the data can have uniform voxel spacing within the transverse plane (e.g., the image slices), and a larger voxel spacing between image slices, the interpolation can only be performed along the axial direction. Additionally, as the data can be acquired utilizing fecal tagging, electronic colon cleansing, which is known in the art, can be performed to remove the tagged colonic materials via an exemplary statistical image segmentation and post-segmentation operation. After the electronic colon cleansing is performed, a clean virtual colon lumen, and a gradient or partial volume (“PV”) layer representing the mucosa or the inner border of the colon wall in a volumetric shell form, can be achieved.

### Exemplary Detection of Initial Polyp Candidates

From the segmented PV layer representing the mucosa or the inner border of the colon wall in a volumetric shell form, computer-aided detection of initial polyp candidates (“IPCs”) (S. Wang, H. Zhu, H. Lu, and Z. Liang (2008), “Volume-based Feature Analysis of Mucosa for Automatic Initial Polyp Detection in Virtual Colonoscopy”, International Journal of Computer Assisted Radiology and Surgery, vol. 3, no. 1-2, 131-142); (H. Zhu, Y. Fan, H. Lu, and Z. Liang, “Improving Initial Polyp Candidate Extraction for CT Colonography”, Physics in Biology and Medicine, vol. 55, no. 3, 2087-2102, (2010), the disclosure of each publication is hereby incorporated by reference in its entirety), can be applied to the volumetric shell to identify suspicious patches or a group of image voxels in the shell. The patch can then be called IPC. From a group of image voxels in the shell, a volume can be extracted which includes image voxels for the associated IPC. The image voxels in the volume associated with an IPC can be used in the feature computation procedure, and in the selection in the volume. The selected features can then be used to reduce the number of FPs in the IPC pool (e.g., the CADe) and further, to differentiate the TPs as malignance or benign (e.g., CADx).

### Exemplary Extraction of Volume of Interest and Volume of Normal

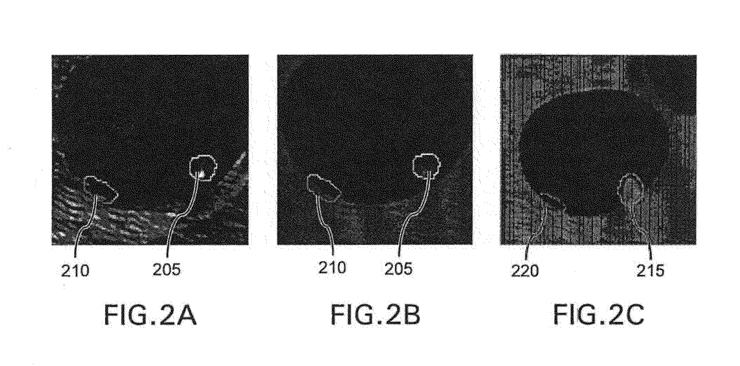

For each IPC identified, its location can be determined on the 2D CT image slices. The IPC borders can be manually outlined on each image slice by repeatedly reviewing it in different window positions and window widths. For example, FIGS. 2(a)-2(c) show exemplary image slices after a manual-based procedure has been performed to outline the IPC borders. By viewing an IPC at different window positions, and window widths, a region of interest (“ROI”) can be drawn on each image slice which can be sufficiently large to cover the entire IPC. FIGS. 2(a) and 2(b) show a pedunculate-shaped polyp (205) and a normal wall mucosa (210) as viewed in different window widths and window positions.

For the borders between the IPC and the lumen (e.g., the ROI-air border), the image contrast is typically very high, and the drawing can be a relatively easy task. Further, the erroneous inclusion of air pixels can be corrected by a known computerized procedure. For the borders between the IPC and the wall (e.g., ROI-tissue border), the image contrast can be limited, and the drawing task is more challenging. According to exemplary embodiments of the present disclosure, the ROI-tissue border can be determined and drawn according to an observed gray value variation by repeated review of an IPC at different window positions and window widths. Some prior knowledge can also be taken into account in the drawing procedure. For example, a ROI-tissue border can often be recognized as having a convex shape and being confined in the mucosa layer. FIG. 2(c) shows an example of a drawn ROI (215) for a sessile polyp, where the ROI-air border includes some air pixels. As noted above, due to the high contrast with the air border, air pixels can subsequently be removed by a computerized procedure, as is well known in the art.

A VOI of normal tissue, or Volume of Normal (“VON”) can be obtained by a similar method of accumulating a number of ROIs of normal tissue or regions of normal (“RON”) on a few 2D CT image slices. A VON can be drawn at a distance proximate to the VOI in the same CTC dataset. The criterion for drawing the RON-tissue borders can be such that they can include normal tissues, and be confined in the mucosa layer and, can have a convex shape. The RON-air borders can have a shape that is either convex or concave depending on its location. For example, if a RON-air border is located on a colon fold, it can have a concave shape. If it is located on the colon wall, it can have a convex shape. The exemplary system, method and computer-accessible medium can also aid in the drawing of RONs such that all the RONs of a VON are consistent to their neighbor RONs. For example, FIG. 2(c) shows an exemplary drawing of a drawn RON (220), where the RON-air border includes some air pixels, which can be corrected later by a known computerized procedure.

By stacking the ROIs and RONs together, initial VOIs and VONs pairs can be obtained. A computerized procedure can be applied to the initial VOIs and VONs to remove the included air voxels. For example, a segmented PV layer, such as disclosed in Wang (Wang S, Li L, Cohen H, Mankes S, Chen J, and Liang Z, “An EM approach to MAP solution of segmenting tissue mixture percentages with application to CT-based virtual colonoscopy”, Medical Physics, 3512: 5787-5798, 2008,) the disclosure of which is hereby incorporated by reference in its entirety, can be used where the air percentage in each of the included voxels has been computed. In order to consider the whole volume of each initial VOI or VON, a global threshold strategy, such as described in Gonzalez (Gonzalez R and Woods R. Digital Image Processing, 2nd ed., Pearson Education, Delhi, India, (2002), the disclosure of which is hereby incorporated by reference in its entirety,) can be employed to remove the included air voxels.

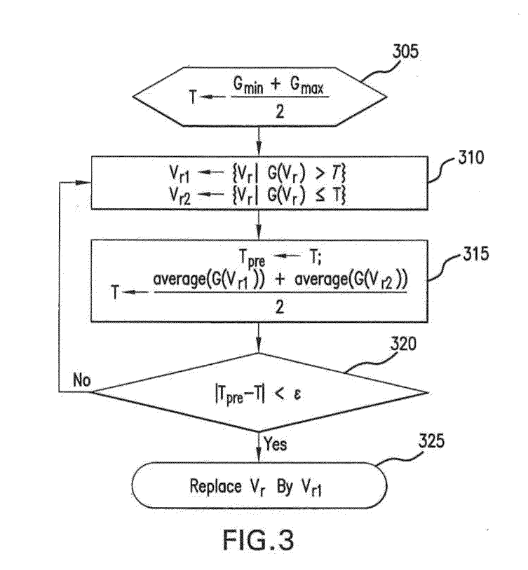

FIG. 3 illustrates a flowchart of an exemplary automatic segmentation procedure based on the global threshold strategy. For example, Vᵣ can be a 3D image that denotes the initial VOI or VON, and G_{(Vr)} can be the set of gray values of voxels in Vᵣ. The histogram of Vᵣ can be obtained from all the voxels contained inside of it (step 305). Gₘᵢₙ and Gₘₐₓ can denote the minimum and maximum gray values in Vᵣ, respectively, and ε can be a predefined small positive number to determine an appropriate point at which to stop the iterative process. After the iterative process, an optimal global threshold T can be obtained and used to segment voxels in Vᵣ into two parts (step 310). With Vᵣ being replaced by Vᵣₗ, the residual voxel's lumen air which can be introduced when outlining the ROIs or RONs slice-by-slice can be removed, and a VOI or VON can be obtained for either a volumetric lesion structure or a normal tissue region.

The procedure for determining the VOI from the group of voxels of a patch, which is initially detected as IPC, can be automated by dilation and erosion operations under constraints of convexity and concavity as described in Zhu et al. (H. Zhu, Z. Liang, M. Barish, P. Pickhardt, J. You, S. Wang, Y. Fan, H. Lu, R. Richards, E. Posniak, and H. Cohen (2010), “Increasing CAD Specificity by Projection Features for CT Colonography”, Medical Physics, vol. 37, no. 4, 1468-1481).

From the obtained volumes of either the VOI or the VON, various features can be extracted for the purposes of CADe and CADx.

### Exemplary 3D Texture Features

A VOI or VON can be treated as a 3D image I, and then texture features can be extracted from the 3D image to form a feature vector for I. For 3D image I, a corresponding 3D gradient image I_{g}, can be computed using, for example, a modified Sobel operator to compute I_{g}, such that it can be applied in a 3D mode. The implemented 3D Sobel kernel in the z-direction can be shown as, for example:

$${h}_{z}^{′} ( : , : , - 1 ) = [ \begin{matrix}1 & 2 & 1 \\ 2 & 4 & 2 \\ 1 & 2 & 1\end{matrix} ] \\ {h}_{z}^{′} ( : , : , 0 ) = [ \begin{matrix}0 & 0 & 0 \\ 0 & 0 & 0 \\ 0 & 0 & 0\end{matrix} ] \\ {h}_{z}^{′} ( : , : , 1 ) = [ \begin{matrix}- 1 & - 2 & - 1 \\ - 2 & - 4 & - 2 \\ - 1 & - 2 & - 1\end{matrix} ] . \qquad ( 1 )$$

For a voxel in image I which can be denoted as I(i, j, k), the derivatives Gₓ(i, j, k), G_{y}(i, j, k) and G_{z}(i, j, k) can be computed in the three orthogonal directions, respectively. The corresponding voxel value in the gradient image I_{g} can be computed according to, for example:

I_{g}(i,j,k)=\sqrt{Gₓ²(i,j,k)+G_{y}²(i,j,k)+G_{z}²(i,j,k)}. (2)

In order to analyze the texture of the volume image I in 3D mode, a model that elaborates the concept of the 3D textures can be used. Utilizing a texture analysis method, such as an extension of the analysis proposed by Haralick et al. (Haralick R and Shanmugam K. “Textural features for image classification”, IEEE Transactions on Systems, Man, and Cybernetics, SMC-3(6): 610-621 (1973), the disclosure of which is hereby incorporated by reference in its entirety), a model can be developed that accounts for the frequency of gray level co-occurrence pairs for a certain distance d along one direction in the 3D space, and records them in a 2D matrix M_{d,θ}, (e.g., gray level co-occurrence matrix (“GLCM”)). The term (x, y, z) can denote the coordinate of a voxel in I, and (x′, y′, z′) can denote another voxel whose Euclidian distance is d along direction θ from (x, y, z). The element (i, j) in the 2D co-occurrence matrix, GLCM, can be computed by, for example:

$${M}_{d , 0} ( i , j ) = {\sum }_{x = 1}^{X} {\sum }_{y = 1}^{Y} {\sum }_{z = 1}^{Z} \{ \begin{matrix}1 & \begin{matrix}if ( x , y , z ) ∈ I , ( {x}^{′} , {y}^{′} , {z}^{′} ) ∈ I , \\ ( x , y , z ) = i and I ( {x}^{′} , {y}^{′} , {z}^{′} ) = j\end{matrix} \\ 0 & otherwise\end{matrix} \qquad ( 3 )$$

where x, y, z represents the dimensions in three axes of I. If the distance d is fixed, the number of co-occurrence matrices that can be generated can be equal to the number of directions used for image I. According to exemplary embodiments of the present disclosure, the distance d can be measured by voxel units, along direction +θ and −θ; the details of which are described below.

Exemplary 3D texture features can include a gradient, a curvature, and a combination of the gradient and the curvature. These exemplary 3D texture features can be created by applying the method proposed by Haralick, but extended into the 3D space, as described in more detail below.

The Haralick model can be used to analyze texture patterns in a 2D gray-level image (e.g., the original density). The basis of Haralick features is the GLCM (e.g., the GLCM shown below). This matrix can be square with dimension N_{g}, where N_{g} can be the number of gray-levels of the density image. Element p(i, j) can be the normalized frequency pixel with value i adjacent to a pixel with value j in a specific direction θ with distance d, which can usually be set to 1. As each pixel has 8 nearest neighbors, there can be four directions, (e.g., 0° (180°), 45° (225°), 90° (270°) and 135° (315°)). The direction 0° can be considered to be the same as direction 180° for the feature calculations. Therefore, only four directions need to be considered. Fourteen initial features are computed from each direction, resulting in a total of 4×14 initial features in each 2D case. For each of the 14 initial features of a direction, the average value and range value over the four directions can be computed, resulting in a total of 2×14 the final features, 14 for the average and 14 for the range. The GLCM representing the so called Haralick features can be written as, for example:

$$GLCM ( θ , d ) = [ \begin{matrix}p ( 1 , 1 ) & p ( 1 , 2 ) & … & p ( 1 , {N}_{g} ) \\ p ( 2 , 1 ) & p ( 2 , 2 ) & … & p ( 2 , {N}_{g} ) \\ ⋮ & ⋮ & ⋱ & ⋮ \\ p ( {N}_{g} , 1 ) & p ( {N}_{g} , 2 ) & … & p ( {N}_{g} , {N}_{g} )\end{matrix} ] \qquad ( 4 )$$

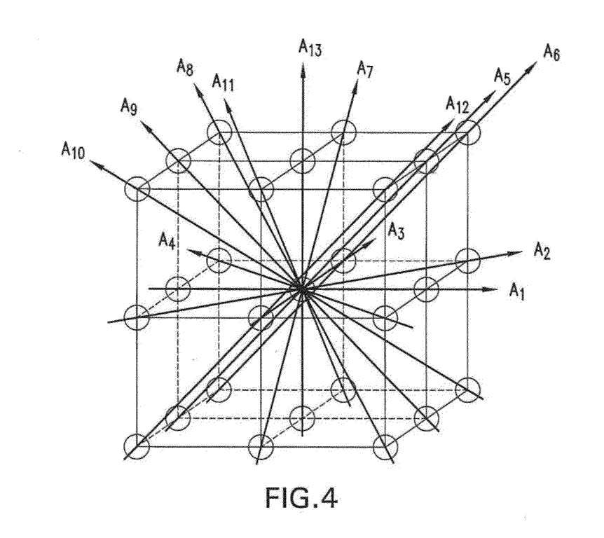

The Haralick method described above can be extended to 3D gray-level image (e.g., the original density). In a 3D image, each voxel can have 26 distance 1 (d=1) neighbors, which can result in 13 directions in the 3D model, such as shown in FIG. 4. The GLCM can be constructed in a similar manner to that of the 2D case. For example, fourteen initial features are computed on each direction. The average and range of each of the 14 initial features can be calculated over the 13 directions, resulting in a total of 2×14 final features, 14 for the average and 14 for the range. For each 3D colon lesion image (e.g., a VOI or VON), a total of 28 texture features can be produced.

The GLCM can capture the correlation information between a pixel and its neighbors in the 2D gray-level density image or in the 3D gray-level density image above. Useful pattern information can also be depicted by the density-gradient/curvature pair, which can produce improved information regarding the lesions because of the higher order representations of the texture patterns, similar to the amplification in microscopy. In addition to the expansion from 2D to 3D, and the amplification, another advantage of the feature calculation above is that the parameter selection step for θ and d, as is known in the art, can be avoided because only one matrix (e.g., the GLCM) will be generated, and no parameter needs to be optimized (both θ and d are determined as described above).

As the orientation of each VOI or VON is unknown, and it is also unknown if the texture features of each VOI or VON are directionally invariant, it is preferable to use a model that is isotropic. In such a case, the derived volumetric features can be invariant regardless of which direction a VOI or VON is oriented. To achieve this, the directions can be uniformly distributed on a unit sphere. In this way, the isotropic trait can be obtained if each feature over all directions is averaged. The resulting directions can be shown in exemplary Table 1 below, which can be represented by vector directions. For each direction in Table 1, directions +θ and −θ can be used to compute a GLCM. Thus, 26 directions, or 13 pairs of directions, can be elaborated which are uniformly distributed on a unit sphere for the GLCM model.

TABLE 1
Directions, represented by vectors used in a 3D GLCM model.

| Serial ID | x | y | Z |
|---|---|---|---|
| 1 | 0 | 0 | 1 |
| 2 | 0 | 1 | 0 |
| 3 | 1 | 0 | 0 |
| 4 | 0 | 1 | 1 |
| 5 | 0 | −1 | 1 |
| 6 | 1 | 1 | 0 |
| 7 | −1 | 1 | 0 |
| 8 | 1 | 0 | 1 |
| 9 | −1 | 0 | 1 |
| 10 | 1 | 1 | 1 |
| 11 | −1 | 1 | 1 |
| 12 | −1 | −1 | 1 |
| 13 | 1 | −1 | 1 |

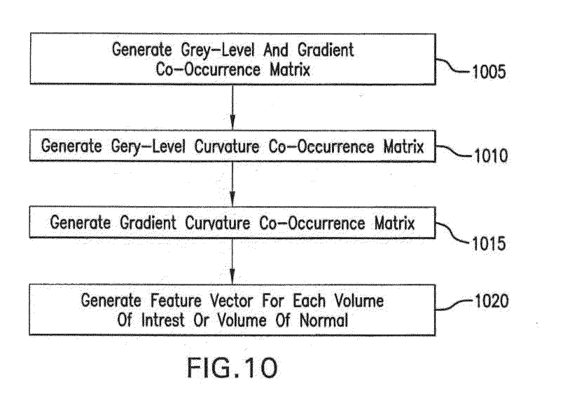

Based on the concept of GLCM derivation in the 3D image, the model can be extended to include a 3D gray level and gradient co-occurrence matrix (“GLGCM”), which can be a derivation of the 3D image (Step 1005 of FIG. 10). The GLGCM describes the mutual pattern between an image and its corresponding gradient image 4. The computation of the GLGCM can be shown as, for example:

$${M}^{′} ( i , j ) = {\sum }_{x = 1}^{X} {\sum }_{y = 1}^{Y} {\sum }_{z = 1}^{Z} \{ \begin{matrix}1 & \begin{matrix}if ( x , y , z ) ∈ I , I ( x , y , z ) = \\ i and {I}_{g} ( x , y , z ) = j\end{matrix} \\ 0 & otherwise .\end{matrix} \qquad ( 5 )$$

The gradient image has a similar size to the original density image. This GLGCM can have a dimension N_{g}×N_{gra} where N_{gra} can be for the number of gradient levels in I_{g}. Element p_{gra} (i,j) can be the normalized frequency of a voxel with value i in density gray-level image and j in corresponding gradient image in the same position. For example:

$$GLGCM = [ \begin{matrix}{p}_{gra} ( 1 , 1 ) & {p}_{gra} ( 1 , 2 ) & … & {p}_{gra} ( 1 , {N}_{g} ) \\ {p}_{gra} ( 2 , 1 ) & {p}_{gra} ( 2 , 2 ) & … & {p}_{gra} ( 2 , {N}_{g} ) \\ ⋮ & ⋮ & ⋱ & ⋮ \\ {p}_{gra} ( {N}_{g} , 1 ) & {p}_{gra} ( {N}_{g} , 2 ) & … & {p}_{gra} ( {N}_{g} , {N}_{g} )\end{matrix} ] \qquad ( 6 )$$

Similar to the GLCM, the GLGCM can capture the second-order statistics as the value of a voxel in I_{g} can be computed from a local neighborhood, which can reflect the inter-voxel relationship in the gradient space. As shown in FIG. 4, a total of 28 features can be computed from the GLGCM; fourteen initial features are computed for each of the 13 directions. For each of the 14 initial features for each direction, the average and range over the 13 directions result in two final features; 2×14 final features.

The dimensions of these co-occurrence matrices of the GLCM and the GLGCM can be very large. According to equation (3), the dimension of the GLCM can reach the maximum gray level in I. The dimension of the GLGCM can be even larger because its dimension can reach the maximum gray level in I, or the maximum gradient in I_{g}, whichever is larger. The CT density value in a VOI or VON can typically range from −1024 HU to 3071HU. Preferably, the original CT value range can be shifted to the range of 1˜4096 in order to guarantee positive i and j in equation (3). Although there is usually only one type of structure in a VOI or VON, and the gray level range can be relatively narrow, the range can still be too wide as compared to the limited dimension of a VOI or VON. A co-occurrence matrix derived based on the above can have the character of a singularity (e.g., contains a lot of zero elements), and can be difficult to manipulate. Therefore, after the shifting operation on the gray values in I, a scaling operation on the gray values of I and I_{g} can be performed to map the values into the same range which can result in two normalized images I′ and I′_{g}, respectively. The scaling operation can have two parameters for the mapping, named rescaling factors S and S_{g}. The rescaling factors S and S_{g} can be determined for an adequate scale from which the textures can be viewed, and can be similar to the magnifying scale of a microscope in biopsy.

After the normalization via the shifting and scaling operations, GLCMs in 13 chosen direction pairs according to equation (3) can be computed (e.g., see Table 1), and 14 square-form GLCMs (corresponding the 14 initial features) from I′ can be obtained. Similarly, a GLGCM from I′ and I′_{g} can be computed according to equation (5).

During In the implementation of extracting the features from GLGCM in the gradient space, there can be a simplification procedure in order to save computation time. For example, the recorded frequency in the GLCM can be employed for an image voxel in the density image to determine the corresponding frequency in the GLGCM for that voxel in the gradient image. Therefore, 14 features can be captured which reflect essentially the same patterns of the 28 features from the GLCM.

In addition to the gradient information, the curvature can be considered to reflect the higher order differentiation of the texture patterns, by which a geometric object deviates from being flat (e.g., a 3D surface). If a surface-like pattern exists inside the 3D volume image, the curvature information can help to improve diagnosis performance. As with the GLGCM, a gray-level curvature co-occurrence matrix (“GLCCM”) can be built (step 1010 of FIG. 10), which can store the density-curvature information, and apply a Haralick model extended into three dimensions to extract pattern features. The key point for calculating curvature is to build up Hessian matrix (H):

$$H = [ \begin{matrix}{I}_{xx} & {I}_{xy} & {I}_{xz} \\ {i}_{xy} & {I}_{yy} & {I}_{yz} \\ {I}_{xz} & {I}_{yz} & {I}_{zz}\end{matrix} ] \qquad ( 7 )$$

where I.. can be the partial derivatives of the grey-level image function I(x,y,z). The 3D Deriche filters can be used to compute the partial derivatives of the image data, where, for example:

f₀(x)=c₀(1+α|x|)e^{-α|x|}

f₁(x)=−c₁xα²e^{-α|x|}

f₂(x)=c₂(1−c₃α|x|)e^{-α|x|} (8)

The normalization coefficients c₀, c₁, c₂, c₃ can be set to, for example:

$${c}_{0} = \frac{{( 1 - {e}^{- α} )}^{2}}{1 + 2 {e}^{- α} α - {e}^{- 2 α}} \\ {c}_{1} = \frac{- {( 1 - {e}^{- α} )}^{3}}{2 {α}^{2} {e}^{- α} ( 1 + {e}^{- α} )} \\ {c}_{2} = \frac{- 2 {( 1 - {e}^{- α} )}^{4}}{1 + 2 {e}^{- α} - 2 {e}^{- 3 α} - {e}^{- 4 α}} \\ {c}_{3} = \frac{( 1 - {e}^{- 2 α} )}{2 α {e}^{- α}} \qquad ( 9 )$$

where α can control the degree of smoothing. The partial derivative can be determined to be, for example:

Iₓₓ=(f₂(x)f₀(y)f₀(z))*I

I_{xy}=(f₁(x)f₁(y)f₀(z))*I (10)

I_{yy}, I_{yz}, I_{xz}, I_{zz} can be determined by substituting the variables in the above equations. Two principal curvatures can be calculated using H, and the Gaussian curvature and the GLCCM can be built up, for example, as:

$$GLCCM = [ \begin{matrix}{p}_{cur} ( 1 , 1 ) & {p}_{cur} ( 1 , 2 ) & … & {p}_{cur} ( 1 , {N}_{cur} ) \\ {p}_{cur} ( 2 , 1 ) & {p}_{cur} ( 2 , 2 ) & … & {p}_{cur} ( 2 , {N}_{cur} ) \\ ⋮ & ⋮ & ⋱ & ⋮ \\ {p}_{cur} ( {N}_{g} , 1 ) & {p}_{cur} ( {N}_{g} , 2 ) & … & {p}_{cur} ( {N}_{g} , {N}_{cur} )\end{matrix} ] \qquad ( 11 )$$

where N_{cur} is the number of curvature levels in the curvature image. By the same description using FIG. 4, fourteen initial features are computed on each direction from the GLCCM. The average and range of each of the 14 initial features can be calculated over the 13 directions, resulting in a total of 2×14 final features; 14 for the average, and 14 for the range.

During the extraction of the features from GLCCM in the curvature space, there can be a simplification procedure in to save computation time. The recorded frequency in the GLCM can be recorded for an image voxel in the density image to find the corresponding frequency in the GLCCM for that voxel in the curvature image. Therefore, 14 features can be captured, which reflect essentially the same patterns of the 28 features from the GLCM.

Both the GLGCM and the GLCCM can be based on the relationship of the high order images and the base grey level image. However, the co-occurrence matrix is constructed as a high order, which can be done by building a gradient curvature co-occurrence matrix (“GCCM”) (step 1015 of FIG. 10) shown below:

$$GCCM = [ \begin{matrix}{p}_{gra\_cur} ( 1 , 1 ) & {p}_{gra\_cur} ( 1 , 2 ) & … & {p}_{gra\_cur} ( 1 , {N}_{cur} ) \\ {p}_{gra\_cur} ( 2 , 1 ) & {p}_{gra\_cur} ( 2 , 2 ) & … & {p}_{gra\_cur} ( 2 , {N}_{cur} ) \\ ⋮ & ⋮ & ⋱ & ⋮ \\ {p}_{gra\_cur} ( {N}_{gra} , 1 ) & {p}_{gra\_cur} ( {N}_{gra} , 2 ) & … & {p}_{gra\_cur} ( {N}_{gra} , {N}_{cur} )\end{matrix} ] \qquad ( 12 )$$

where N_{gra} and N_{cur} are the number of gradient and curvature levels in I_{gra} and I_{cur} respectively. In this case, similar to the simplification implementation for the models of the GLGCM and the GLCCM, a total of 14 texture features can be calculated based on the GCCM. The recorded frequency in the GLGCM can be recorded for an image voxel in the gradient image to find the corresponding frequency in the GCCM for that voxel in the curvature image. Therefore, 14 features can be captured which reflect essentially the same patterns of the 28 features from the GLGCM.
The GLCM, GLGCM, GLCCM, and GCCM, for each VOI or VON, can represent our texture model of the volume. From this texture model, texture features can be derived to perform the CADe and CADx tasks. For example, from the original GLCM of Haralick et al, 14 features can be computed, as suggested by Haralick et al. (Haralick R and Shanmugam K., “Textural features for image classification”, IEEE Transactions on Systems, Man, and Cybernetics, SMC-3(6): 610-621 (1973)). In one preferred embodiment, the 12 features can include Angular Second Moment (e.g., Energy), Contrast, Correlation, Sum of Squares (e.g., Variance), Inverse Difference Moment, Sum Average, Sum Variance, Sum Entropy, Entropy, Difference Variance, Difference Entropy, Information Measures of Correlation in two forms, and Maximal Correlation Coefficient. Equations for the computation of these features can be seen in the following equations, and in Table 2, where p(i, j) can be the (i, j)th entry in a normalized gray-tone spatial-dependence matrix. N_{g} can be the number of distinct gray levels in the quantized image. Σᵢ and Σⱼ are Σᵢ₌₁^{N}^{_{g}} and Σⱼ₌₁^{N}^{_{g}}, respectively.

pₓ(i)=Σᵢ₌₁^{N}^{_{g}}p(i,j).

p_{y}(j)=Σⱼ₌₁^{N}^{_{g}}p(i,j).

μₓ, μ_{y}, σₓ and σ_{y} can be the means and standard deviations of pₓ and p_{y}.

$${p}_{x + y} ( k ) = {{Σ}_{i} {Σ}_{j}}_{i + j = k} p ( i , j ) , k = 2 , 3 , … , 2 {N}_{g}$$

$${p}_{x + y} ( k ) = {{Σ}_{i} {Σ}_{j}}_{\vert i - j \vert = k} p ( i , j ) , k = 0 , 1 , … , {N}_{g} - 1$$

$$HXY = - {Σ}_{i} {Σ}_{j} p ( i , j ) log ( p ( i , j ) ) , \\ HXY 1 = - {Σ}_{i} {Σ}_{j} p ( i , j ) log ( {p}_{x} ( i ) {p}_{y} ( j ) ) .$$

HX and HY are entropies of pₓ and p_{y}.

$$HXY 2 = - {\sum }_{i} {\sum }_{j} {p}_{x} ( i ) {p}_{y} ( j ) log \{ {p}_{z} ( i ) {p}_{y} ( j ) \}$$

$$Q ( i , j ) = {\sum }_{k} \frac{p ( i , k ) p ( j , k )}{{p}_{x} ( i ) {p}_{y} ( k )}$$

TABLE 2
Equations of 14 exemplary Haralick features.

| ID | Name | Equation |
|---|---|---|
| 1 | Angular Second Moment (Energy) | ${f}_{1} = {\sum }_{i}^{ } {\sum }_{j}^{ } {\{ p ( i , j ) \}}^{2}$ |
| 2 | Contrast | ${f}_{2} = {\sum }_{n = 0}^{{N}_{g} - 1} {n}^{2} \{ {{\sum }_{i}^{ } {\sum }_{j}^{ }}_{\vert i - j \vert = n} p ( i , j ) \}$ |
| 3 | Correlation | ${f}_{3} = \frac{{\sum }_{i}^{ } {\sum }_{j}^{ } ( ij ) p ( i , j ) - {μ}_{x} {μ}_{y}}{{σ}_{x} {σ}_{y}}$ |
| 4 | Variance (Sum of Squares) | ${f}_{4} = {\sum }_{i}^{ } {\sum }_{j}^{ } {( i - μ )}^{2} p ( i , j )$ |
| 5 | Inverse Difference Moment | ${f}_{5} = {\sum }_{i}^{ } {\sum }_{j}^{ } \frac{p ( i , j )}{1 + {( i - j )}^{2}}$ |
| 6 | Sum Average | ${f}_{6} = {\sum }_{i = 2}^{2 {N}_{g}} {ip}_{x + y} ( i )$ |
| 7 | Sum Variance | ${f}_{7} = {\sum }_{i = 2}^{2 {N}_{g}} {( i - {f}_{8} )}^{2} {p}_{x + y} ( i )$ |
| 8 | Sum Entropy | ${f}_{8} = - {\sum }_{i = 2}^{2 {N}_{g}} {p}_{x + y} ( i ) log \{ {p}_{x + y} ( i ) \}$ |
| 9 | Entropy | ${f}_{9} = - {\sum }_{i}^{ } {\sum }_{j}^{ } p ( i , j ) log ( p ( i , j ) )$ |
| 10 | Difference Entropy | f₁₀ = variance of p_{x-y} |
| 11 | Difference Variance | ${f}_{11} = - {\sum }_{i = 0}^{{N}_{g} - 1} {p}_{x - y} ( i ) log \{ {p}_{x - y} ( i ) \}$ |
| 12 | Information Measures of Correlation | ${f}_{12} = \frac{HXY - HXY 1}{max \{ HX , HY \}}$ |
| 13 | Information Measures of Correlation | f₁₃ = (1 − exp[−2.0(HXY2 − HXY)])^{1/2} |
| 14 | Maximal Correlation Coefficient | f₁₄ = (Second largest eigenvalue of Q)^{1/2} |

The above 14 features can be the initial features on a direction. For 13 directions in 3D space, there can be a total of 13×14 initial features. The initial features' average and range over the 13 directions can be computed, resulting in 2×14 final features; 14 for the average and 14 for the range. As previously discussed, the averaging over uniformly distributed 3D directions can produce the isotropic trait for each feature from the GLCMs. Two first-order statistical features can also be computed (e.g., mean and variance), directly from the volume data of VOI or VON. Thus 2×14+2=30 features can be computed from the GLCM.

Similarly 2×14 final features from the GLGCM, 2×14 final features from the GLCCM, and 2×14 final features from the GCCM can be computed. Concatenating these features together, a 114-feature vector can be formed for each VOI or VON (step 1020 of FIG. 10). Using the simplified implementation procedure, the concatenated feature vector has a dimension of 30+3×14=72. The use of these volumetric texture features for CADe and CADx of colonic polyps will be discussed in further detail below. For CADe purpose, these features will be used to remove the FPs in the IPC pool. Then the remaining IPCs will be treated as TPs or polyps. For CADx purpose, these features will be used to differentiate polyp types as hyperplastic and adenomas, or, more generally, as benign and malignance.

### Exemplary CTC Database Evaluation

The exemplary texture model with derived volumetric features was tested in a CTC database that includes 67 patients who have each undergone CTC screening with FOC follow-ups. Each patient followed a one-day low-residue diet with oral contrast for fecal tagging, and underwent two CTC scans in both supine and prone positions. Multi-detector (e.g., 4- and 8-MDCT) scanners were used and 134 CTC scans were produced. The scanning protocols included mAs modulation in the range of 120-216 mA with kVp of 120-140 values, 1.25-2.5 mm collimation, and reconstruction interval of 1 mm. The scanners rotated at a speed of 0.5 seconds of rotation. A total of 119 polyps and masses, sized in the range of 4-30 mm, were confirmed by both FOC and CTC. The scans were considered at two positions for each patient as two different scans, which resulted in 134 CTC scans and 238 polypoid cases. To avoid the interference from the oral contrast tagging, 47 cases that were sized less than 5 mm and almost totally buried in the contrasted materials were excluded from the study. Thus a database with 191 lesions (e.g., polyps and masses) was created from the 134 CTC scans. As described above, both manual and automated procedures outlined 191 VOI cases corresponding to the true polyps and masses or true positives in the 134 CTC scans. The additional 191 VONs from normal tissue regions were extracted for comparison purpose, resulted in 382 cases in total to evaluate the proposed volumetric features. Given the biopsy results in the FOC screening, the detailed information of the database of 382 cases is shown in Table 2. The numbers of hyperplastic (“H”), tubular adenoma (“Ta”), tubulovillous adenoma (“Va”) and adenocarcinoma (“A”) polyps or masses was 56, 94, 34 and 7, respectively. The trend of the risk rate is also shown by the right column in Table 3.

TABLE 3
Data details of the database for evaluation.

| Pathology Type | Abbreviation | Cases | Risk rate |
|---|---|---|---|
| Adenocarcinoma Tubulovillous adenoma Tubular adenoma Hyperplastic Normal | A Va Ta H Norm | 7 34 94 56 191 | [symbol] |
| Total | 5 | 382 | |

### Exemplary CADe Evaluation

To ensure a high sensitivity rate, a typical CADe pipeline for CTC can generate a large number of IPCs, which can be a mixture of TPs and FPs. A variety of filters which utilize some geometric or textural features, have been designed to reduce the FPs. To test the ability of the proposed volumetric texture features in FP reduction, the 191 VOIs and 191 VONs were treated as IPCs from the initial operation of a CADe pipeline or CAD of IPCs (S. Wang, H. Zhu, H. Lu, and Z. Liang (2008), “Volume-based Feature Analysis of Mucosa for Automatic Initial Polyp Detection in Virtual Colonoscopy”, International Journal of Computer Assisted Radiology and Surgery, vol. 3, no. 1-2, 131-142] [H. Zhu, Y. Fan, H. Lu, and Z. Liang, “Improving Initial Polyp Candidate Extraction for CT Colonography”, Physics in Biology and Medicine, vol. 55, no. 3, 2087-2102, (2010)), where VOIs can reflect the TPs and VONs can reflect the FPs. Two classifiers of support vector machine (“SVM”) and linear discriminant analysis (“LDA”) were used to perform the task of FP reduction in the IPC pool. All the derived feature vectors were randomly assembled, and were trained and tested under a two-fold cross-validation strategy. For each fold, all features were randomly assigned to two sets d₀ and d₁, such that both sets were equally sized. Then training was performed on dₒ and testing was performed on d₁, followed by training on d₁ and testing on d₀. This testing scheme has the advantage in that the sample size of the training and test sets is large, and each feature vector is used for both training and testing on each fold. The two-fold cross-validation was repeated 50 times. The average sensitivity and specificity of the classification can indicate the ability of the proposed method to reduce the number of FPs.

### Exemplary CADx Evaluation

In order to discriminate among the pathology types of the polyps using the proposed model (FIG. 1, step 135), the feature vectors of VOIs were grouped into four classes, labeled by H, Ta, Va, and A. The four types of polyps can be a series of pathologically-defined colon tissue types with increasing risk of turning into caneinomous (e.g., as seen in Table 3 above).

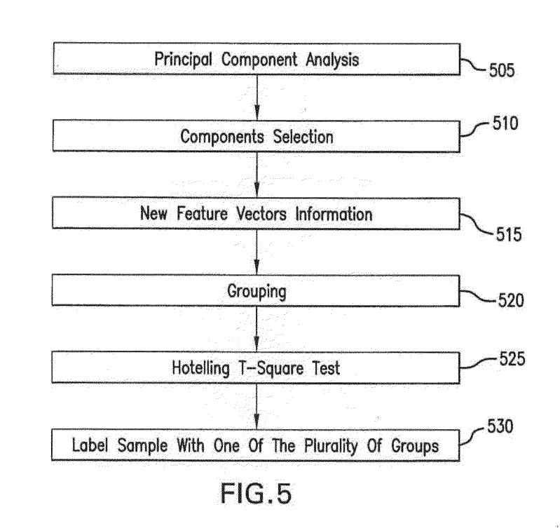

FIG. 5 shows an exemplary evaluation procedure designed to test whether there are significant differences when using the proposed volumetric texture features. For the purpose of completeness, the feature vectors of VONs, labeled as Norm, were included in the scheme. To relieve the computation workload, and address the problem of feature numbers larger than the sample numbers (e.g., over 100 features vs. 7 adenocarcinomas), the principal component analysis (“PCA”) (procedure 505), such as described in Jolliffe (Jolliffe I. Principal Component Analysis, Series: Springer Series in Statistics, 2nd ed., Springer, N.Y., XXIX, 487, pp. 28 (2002), the disclosure of which is hereby incorporated by reference in its entirety), can be employed to obtain the orthogonal distributed components. Then, the 7 most scored principal components can be selected to transfer the original 114-feature vectors, or 72-feature vectors using the simplified implementation procedure, into new 7-feature vectors (procedure 510). The features in each newly formed vector can be the linear combination of the original features with the chosen PCA components coefficients (procedure 515), and groups (e.g., five groups) can be formed (procedure 520). Then significance testing among the five groups using a Hotelling T-square test, as described in Hotelling (Hotelling H, “The generalization of Student's ratio,” Annals of Mathematical Statistics, 2(3): 360-378 (1931)), can be processed based on the newly formed vectors (procedure 525). The Hotelling T-square test can be a generalization of the univariate student T-test, which has been widely used for multivariate problem which tests whether the mean values of the multi-variate distribution are same. According to exemplary embodiments of the present disclosure, each sample can be a 7-variate vector that can be labeled with one of the groups' tags (e.g., Norm, H, Ta, Va and A) (procedure 530). The results of the Hotelling T-square test can show whether there are significant differences between each pair from the five groups based on the proposed model.

### Exemplary Preprocessing Results

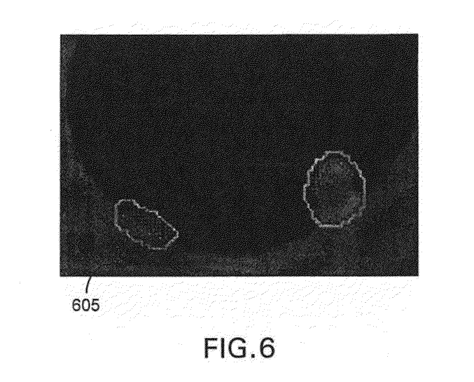

After the interpolation procedure, an average of 725±91 slices was obtained for all CTC scans, and a cubic voxel size of 0.7174±0.0744 min was used. In the extraction of VOIs and VONs, the parameter s used to stop the iteration was 10⁻⁸. The global threshold T was set at 452.83±59.09 HU for the entire database. FIG. 6 shows an exemplary image slice showing the result of the global thresholding segmentation strategy, which corresponds to the manually outlined result as shown in FIG. 2(c). Area 605 can be labeled as an air region and can be removed after the global thresholding segmentation process. For all 382 cases of extracted VOIs and VONs, the GLCMs (d=1) and the GLGCMs were computed after all voxels in I, I_{gra} and I_{cur} were mapped into the level range of [1, 32]. Then a 114-feature vector, or 72-feature vector using the simplified implementation procedure, was formed for each volume data (e.g., VOI or VON) according to the exemplary procedure above.

### Exemplary FP Reduction or CADe Results

A suitable SVM package for use in the present systems and methods is described in Chang. (Chang C and Lin C. “LIBSVM: A library for support vector machines”, ACM Transactions on Intelligent Systems and Technology, 2(27):1-27, 2011), the disclosure of which is hereby incorporated by reference in its entirety). A guide was followed to use a grid search to select the best-fit parameters. The LDA used was implemented by the R CARN package [e.g., R CARN package Online]. For the purpose of testing the proposed model in a CADe pipeline for FP reduction, the 382 vectors extracted from VOIs and VONs were randomly sorted, and then randomly split in half. The two-fold cross-validation was implemented to obtain the sensitivity and the corresponding specificity. The random grouping and two-fold cross-validation procedures were iteratively repeated 50 times. The final results were drawn on the average of the 50 iterations, as shown in Table 4 below.

TABLE 4
Classification result of SVM and LDA.

| | Sensitivity | Specificity |
|---|---|---|
| SVM | (96.90 ± 2.18)% | (99.16 ± 1.02)% |
| LDA | (99.87 ± 0.35)% | (99.93 ± 0.27)% |

### Exemplary CADx Results

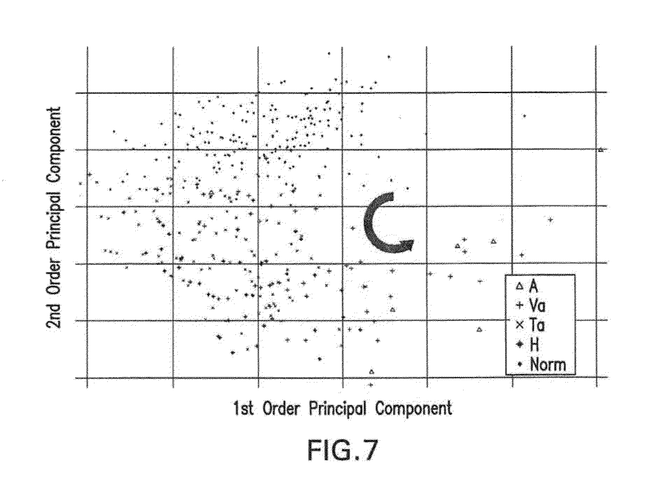

According to the evaluation method of FIG. 5, four types of VOI-derived feature vectors, namely A, Va, Ta and H, plus the feature vectors of normal tissue labeled as Norm underwent a PCA procedure. The first- and second-order principal components can be seen plotted in the graph of FIG. 7.

Referring to FIG. 7, visually the VONs and VOIs are separated very well. As for the four polyp types in VOIs, there is no distinct boundary between each paired type. However, the clustering trend of each polyp type can be seen. Also it can be seen that there is an evolving locus from normal to adenocarcimous, as shown by 705. The sequence of VOI types along the evolving locus coincides with the order of their risk rates (e.g., the right column of Table 2).

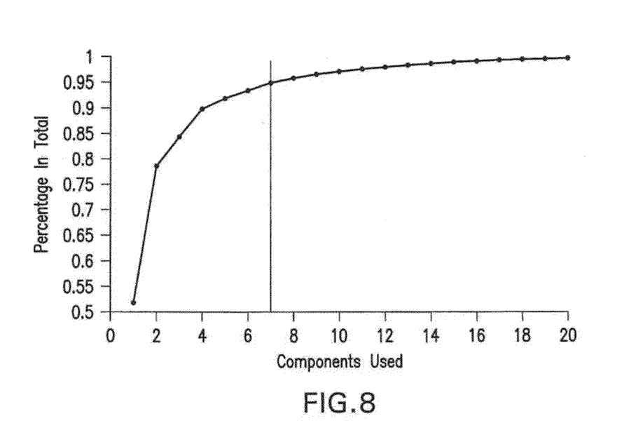

According to the accumulative variance percentage curve shown in FIG. 8, the most scored 7 principal components method was chosen to transform the original 114-feature vectors, or 72-feature vectors using the simplified implementation, into 7-feature vectors. This process can account for 94.9% of the total accumulated. PCA components variance. After the transformation, the Hotelling T-square test can be performed between each pair from the five groups labeled as Norm, H, Ta, Va and A, respectively. The results of which are set forth in Table 5 below.

TABLE 5
Paired Hotelling T-square tests of the 5 groups.

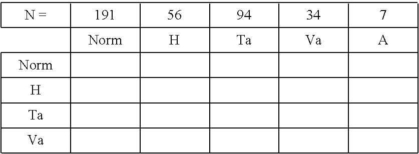

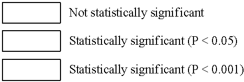

As is evident from the above, there is no significant difference between groups H and Ta. However, significant difference does exist between groups of A and Va (p<0.05), A and H/Ta (p<0.001), and Va and H/Ta (p<0.001). The Norm group is significantly different from each of the four polypoid groups. This can also be seen in the CADe results in Table 3 above.

To further explore the above case of no significant difference between groups H and Ta, more Hotelling T-square tests were performed as the number of principal components in FIG. 8 increased. This is possible because the sample size of these two groups is larger than 7. The two groups reached a significant difference when the number of the principal components increased to 20.

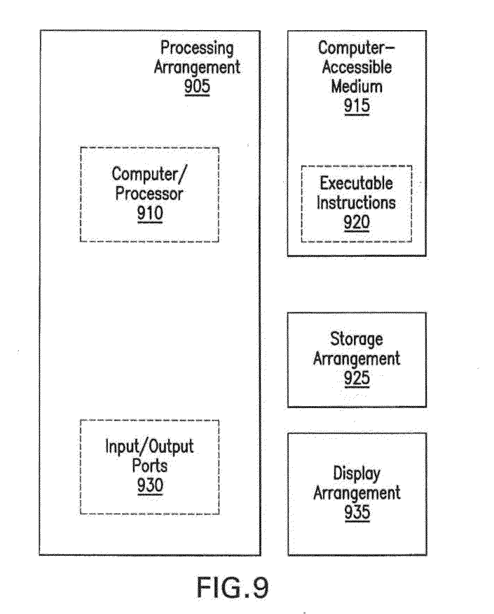

FIG. 9 shows a block diagram of an exemplary embodiment of a system suitable for practicing the process described above for computer-aided detection and diagnosis of polyps according to the present disclosure. For example, exemplary procedures in accordance with the present disclosure described herein can be performed by a processing arrangement and/or a computing arrangement 905. Such processing/computing arrangement 905 can be, for example, entirely or a part of, or include, but not limited to, a computer/processor 910 that can include, for example one or more microprocessors or computer processors, and use instructions stored on a computer-accessible medium (e.g., RAM, ROM, hard drive, or other storage device).

As shown in FIG. 9, for example, a computer-accessible medium 915 (e.g., as described herein above, a storage device such as a hard disk, floppy disk, memory stick, CD-ROM, RAM, ROM, etc., or a collection thereof) can be provided and is in communication with the processing arrangement 905. The computer-accessible medium 915 can contain executable instructions 920 thereon. In addition or alternatively, a storage arrangement 925 can be provided separately from the computer-accessible medium 915, which can provide the instructions to the processing arrangement 905 so as to configure the processing arrangement to execute certain exemplary procedures, processes and methods, as described herein above, for example.

Further, the exemplary processing arrangement 905 can be provided with or include an input/output arrangement 930, which can include, for example, a wired network, a wireless network, the internet, an intranet, a data collection probe, a sensor, etc. As shown in FIG. 9, the exemplary processing arrangement 905 can be in communication with an exemplary display arrangement 935, which, according to certain exemplary embodiments of the present disclosure, can be a touch-screen configured for inputting information to the processing arrangement in addition to outputting information from the processing arrangement, for example. Further, the exemplary display 935 and/or a storage arrangement 925 can be used to display and/or store data in a user-accessible format and/or user-readable format.

The term “about,” as used herein, should generally be understood to refer to both the corresponding number and a range of numbers. Moreover, all numerical ranges herein should be understood to include each whole integer within the range.

While illustrative embodiments of the disclosure are disclosed herein, it will be appreciated that numerous modifications and other embodiments may be devised by those skilled in the art. For example, the features for the various embodiments can be used in other embodiments. Therefore, it will be understood that the appended claims are intended to cover all such modifications and embodiments that come within the spirit and scope of the present disclosure.

1. A computer-based method for diagnosing a region of interest within an anatomical structure, comprising:
receiving a 3D volumetric representation of the anatomical structure;
identifying at least one volume of interest and volume of normal of the anatomical structure;
determining a density, a gradient and a curvature of the volume of interest;
generating a first feature set based on the density, the gradient and the curvature; and
comparing the first feature set to a second feature set to diagnose the region of interest to at least one of a plurality of pathology types.

2. The computer-based method of claim 1, wherein the generation of the first feature set comprises combining the density, the gradient and the curvature to produce the first feature set.

3. The computer-based method of claim 2, wherein at least one of the density, the gradient or the curvature is determined using a 3D Haralick model.

4. The computer-based method of claim 3, wherein the gradient is determined using a gray-level gradient co-occurrence matrix.

5. The computer-based method of claim 3, wherein the curvature is determined using a gray-level curvature co-occurrence matrix.

6. The computer-based method of claim 3, wherein the density is determined using a gray-level co-occurrence matrix.

7. The computer-based method of claim 1, wherein the second feature set is generated by manually analyzing a plurality of regions of interest.

8. The computer-based method of claim 1, wherein the region of interest is a polyp.

9. The computer-based method of claim 1, further comprising detecting the region of interest.

10. The computer-based method of claim 9, wherein the region of interest is diagnosed only if the region of interest is detected to be a polyp.

11. The computer-based method of claim 9, wherein the detection comprises:
detecting initial polyp candidates on the colon wall; and
extracting the volume of interest from the initial polyp candidates;

12. The computer-based method of claim 9, wherein the detection comprises comparing the volume of interest to the volume of normal in the 3D volumetric representation of the anatomical structure.

13. The computer-based method of claim 11, wherein the initial polyp candidates comprises a group of image voxels on the mucosa layer of the colon wall, and the volume of interest comprises the group of image voxels on the mucosa layer of the colon wall and a plurality of additional voxels not associated with the initial polyp candidates.

14. The computer-based method of claim 1, further comprising generating the 3D volumetric representation of the anatomical structure using an in-vivo imaging method.

15. The computer-based method of claim 1, wherein the first feature set and the second feature set comprise at least 50 features.

16. The computer-based method of claim 1, wherein the first feature set is compared to the second feature set using a support vector machine.

17. The computer-based method of claim 1, further comprising receiving 2D imaging information of the anatomical structure, and converting the 2D imaging information into the 3D volumetric representation of the anatomical structure.

18. The computer-based method of claim 17, wherein the 2D imaging information is generated using computed tomography.

19. A non-transitory computer-accessible medium including a set of instructions executable by a processor, the set of instructions operable to:
receive a 3D volumetric representation of an anatomical structure;
identify at least one volume of interest and volume of normal of the anatomical structure;
determine a density, a gradient and a curvature of the volume of interest;
generate a first feature set based on the density, the gradient and the curvature; and
compare the first feature set to a second feature set to diagnose a region of interest to at least one of a plurality of pathology types.

20. A system for diagnosing a region of interest within an anatomical structure, comprising:
a processor;
software executing on the processor to receive a 3D volumetric representation of the anatomical structure;
software executing on the processor to identify at least one volume of interest and volume of normal of the anatomical structure;
software executing on the processor to determine a density, a gradient and a curvature of the volume of interest;
software executing on the processor to generate a first feature set based on the density, the gradient and the curvature; and
software executing on the processor to compare the first feature set to a second feature set to diagnose the region of interest to at least one of a plurality of pathology types.

21-29. (canceled)
